(19) United States
(12) Patent Application Publication
(10) Pub. No.: US 2026/0162787 A1
(43) Pub. Date: Jun. 11, 2026

(54) SECURE DIAGNOSTIC RESULT RETURNS IN RESOURCE-CONSTRAINED COMPUTING ENVIRONMENTS

(71) Applicant: Cooper Smith Global, LLC, Austin, TX (US)

(72) Inventors: Tyler R. Smith, Pensacola, FL (US); Hannah J. Klein, Austin, TX (US)

(21) Appl. No.: 19/414,166

(22) Filed: Dec. 9, 2025

Related U.S. Application Data
(60) Provisional application No. 63/729,792, filed on Dec. 9, 2024.

(51) Int. Cl. G16H 10/60; G16H 10/40; G16H 40/67

(52) U.S. Cl. CPC G16H 10/60; G16H 10/40; G16H 40/67

(57) ABSTRACT

Provided is a process to securely return diagnostic results in a resource constrained computing environment. The process includes: receiving, at a server system, via a session-based, carrier signaling channel, as part of a session, an identifier associated with a medical record in a medical records data repository, the identifier having been sent by a user's phone; retrieving, with the server system, based on the identifier, the medical record; and sending, with the server system, via the session-based, carrier signaling channel, in the session, a text indication of a test result in the medical record to the user's phone.

### CROSS-REFERENCE TO RELATED APPLICATIONS

This application claims priority to U.S. Provisional Application No. 63/729,792, filed Dec. 9, 2024, the subject matter of which is incorporated herein by reference in its entirety.

### BACKGROUND

### 1. Field

The present disclosure relates generally to secure diagnostic result returns in resource-constrained computing environments.

### 2. Description of the Related Art

Timely results from diagnostic medical tests, particularly for viral infections, are helpful for several reasons. They facilitate early diagnosis, allowing treatment to begin promptly, which can significantly improve outcomes, especially when interventions are most effective shortly after infection. Quick results also help prevent the spread of contagious viruses by helping individuals to take immediate precautions, such as isolating or wearing masks, reducing transmission risks. Patients benefit from reduced anxiety and uncertainty when results are delivered quickly, and healthcare systems can better allocate resources, focusing on those who need care most urgently. Accurate, timely diagnoses support informed decision-making, helping individuals and healthcare providers determine the best course of action, whether it involves quarantine, treatment, or returning to normal activities. Further, rapid test results aid in tracking infection patterns, guiding effective outbreak management, and preventing complications, particularly in vulnerable populations. Generally, the prompt delivery of test results enhances individual care and strengthens community health.

### SUMMARY

The following is a non-exhaustive listing of some aspects of the present techniques. These and other aspects are described in the following disclosure.

Some aspects include a process to securely return diagnostic results in a resource constrained computing environment. The process includes: receiving, at a server system, via a session-based, carrier signaling channel, as part of a session, an identifier associated with a medical record in a medical records data repository, the identifier having been sent by a user's phone; retrieving, with the server system, based on the identifier, the medical record; and sending, with the server system, via the session-based, carrier signaling channel, in the session, a text indication of a test result in the medical record to the user's phone.

Some aspects include a tangible, non-transitory, machine-readable medium storing instructions that when executed by a data processing apparatus cause the data processing apparatus to perform operations including the above-mentioned process.

Some aspects include a system, including: one or more processors; and memory storing instructions that when executed by the processors cause the processors to effectuate operations of the above-mentioned process.

### BRIEF DESCRIPTION OF THE DRAWINGS

The above-mentioned aspects and other aspects of the present techniques will be better understood when the present application is read in view of the following figures in which like numbers indicate similar or identical elements:

FIG. 1 is a block diagram illustrating an example of a computing environment with a Viral Load Results Return (VLRR) system in which the present techniques may be implemented, in accordance with some embodiments;

FIG. 2 illustrates the operation Service Discovery Platform in element C of FIG. 1, in accordance with some embodiments;

FIG. 3 depicts an example workflow to use features of FIG. 1, in accordance with some embodiments;

FIG. 4 illustrates the operation of element D in FIG. 1, in accordance with some embodiments;

FIG. 5 illustrates an example interface for the Home Monitoring and Evaluation Dashboard of FIG. 1, in accordance with some embodiments;

FIG. 6 illustrates a workflow for registering a client in the VLRR system of FIG. 1, in accordance with some embodiments;

FIG. 7 is an example of a computing environment in which some embodiments may be implemented;

FIG. 8 is an example of a process that may be executed by some embodiments; and

FIG. 9 illustrates an example computing device by which the VLLR system may be implemented.

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION OF CERTAIN EMBODIMENTS

To mitigate the problems described herein, the inventors had to both invent solutions and, in some cases just as importantly, recognize problems overlooked (or not yet foreseen) by others in the fields of public health and computer science. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Delivering medical diagnostic test results via mobile devices can be highly effective even in less wealthy countries due to the widespread proliferation of mobile phones, which have become accessible across diverse socioeconomic groups. Mobile technology often bypasses the need for traditional infrastructure, such as physical mail or landline networks, making it particularly valuable in regions with limited healthcare facilities. Since mobile phones are ubiquitous and often the primary mode of communication, they facilitate quick and direct delivery of results to individuals, often affording timely action and reducing delays caused by logistical challenges. This approach not only enhances individual healthcare outcomes by facilitating immediate access to information but also strengthens public health efforts by improving communication and facilitating rapid responses to potential outbreaks.

Native applications, including web browsers, are often not ideal for delivering medical test results in less wealthy countries because they generally presume a smart phone (rather than just a feature phone) and require users to download and install the application, which can introduce friction and may deter individuals from accessing the service delivering results. This process can be particularly challenging in areas with limited internet bandwidth, where downloading an app may be slow, expensive, or even impossible for those with restricted data plans. Additionally, native apps often require regular updates to function properly, further increasing data usage and creating potential barriers for users who lack consistent internet access. These challenges make simpler, more accessible solutions, such as web-based platforms, SMS, or lightweight messaging services, better suited to ensuring widespread and equitable access to critical health information. Indeed, in some cases the patient's mobile computing device may not have the ability to install and execute native applications and may be limited to text based messaging and voice calls.

That said, in many cases, SMS (short message service) is not ideal for delivering sensitive medical test results either, because the information remains stored in the user's phone within the chat history, where it can be accessed by others who may use or gain unauthorized access to the device, and because the service can entail costs in some cases that users prefer to avoid. The potential lack of privacy and security poses a significant risk, particularly in situations where confidentiality is crucial, such as in cases involving stigmatized illnesses or sensitive health conditions. Unlike secure communication methods, SMS does not offer encryption or the ability to control message visibility, leaving the data vulnerable to unintended exposure. This limitation can make SMS a less suitable option for handling sensitive health information in a way that ensures privacy and compliance with data protection standards.

None of the proceedings should be taken to suggest that any technique is disclaimed or disavowed, as the present techniques may be used in conjunction with these approaches, for instance using native apps or SMS. Further discussion of other trade-offs or challenges with various approaches should not be read to imply that any technique is disclaimed or disavowed, nor should discussion of any advantages of any approaches be taken to imply that embodiments are limited to systems that provide those advantages.

In some embodiments, Unstructured Supplementary Service Data (USSD) may be employed as a mechanism for distributing medical diagnostic results, e.g., to patients in lower-income countries using widely available mobile phones, such as smart phones or feature phones. USSD may operate through a session-based communication protocol over GSM (Global System for Mobile Communications) networks, allowing users to interact with embodiments like those described below via shortcodes. A user may initiate a session by dialing a specific USSD code, such as *123#, which triggers a request to the application server. The server may process the request and transmit a menu or textual response back to the user in real-time. This interactive session may persist until terminated by the user or by a timeout event defined by the protocol.

To distribute medical diagnostic results, some embodiments may integrate a USSD-based interface with a backend health information management system. After diagnostic data is processed, the system may associate the results with a unique identifier for each patient, such as a phone number or an assigned patient ID. Patients may enter a predefined USSD code and authenticate themselves, possibly through a numeric PIN or a one-time password delivered via a secondary channel. Upon successful authentication, the USSD application may query the database for the associated medical results and display the information directly to the patient's device in a series of brief text responses within the session.

Unlike SMS, USSD may not require the storage of messages on the mobile device or within the network, as the data exchanged exists only during the live session. This ephemeral nature of USSD communication may enhance data security, reducing the risk of unauthorized access to sensitive medical information that might otherwise remain stored on the device. Moreover, USSD may operate without the need for a subscriber to have an active SMS balance, as it typically works independently of conventional messaging charges, relying instead on session-based billing or flat-rate service models.

In some embodiments, USSD may also support multiple language options by dynamically adjusting the session content based on the user's selected language preference, facilitating accessibility for diverse populations. Additionally, because USSD relies on GSM standards and operates effectively on basic mobile phones, it may offer broader compatibility in regions where smartphones and internet connectivity are less prevalent.

Developers implementing USSD-based systems for this purpose may incorporate features such as hierarchical menus for navigating results, integration with health data anonymization techniques to protect patient privacy, and session expiration safeguards to further secure sensitive information. The system may also include support for feedback loops, allowing patients to request clarification or additional information about their results, potentially through a follow-up session initiated by a subsequent USSD code.

In some embodiments, USSD session-oriented protocol may involve communication between the mobile device, the Mobile Switching Center (MSC), and an application server, such as one hosted by a network operator or third-party service provider.

When a user initiates a USSD session, they may dial a USSD string, such as *123#. This input may be transmitted to the serving MSC using a signaling message, formatted according to the GSM MAP (Mobile Application Part) protocol. The MSC, in some embodiments, identifies the request as a USSD service and routes it to a USSD gateway or directly to the application server, which may be connected via a signaling link such as SS7 (Signaling System No. 7) or over an IP-based network using SIGTRAN (Signaling Transport).

Upon receiving the USSD request, the application server may process the input and generate an appropriate response. The server may use pre-defined logic, database queries, or external application programming interfaces (APIs) to determine the response content. This response is sent back through the USSD gateway to the MSC, which forwards it to the mobile device in real-time.

USSD communication, in some embodiments, occurs within the context of a live session, meaning the connection remains open for a series of exchanges between the mobile device and the application server. The protocol may support a request-response interaction model where each input from the user elicits a response from the server. For example, a hierarchical menu system may present options to the user, such as “Press 1 for Latest Results,” “Press 2 for Past Results,” and so on. Each user input prompts a new query to the server, which dynamically generates and returns the next menu or information.

Sessions, in some embodiments, are time-sensitive and may terminate automatically after a predefined period of inactivity or upon explicit termination by the user or application. This ephemeral nature is expected to reduct the risk of data lingering in transit or storage. The data exchange is may be limited to 182 characters per USSD message in some cases, though multi-part messages may be supported by the application logic to provide longer responses.

The protocol, in some embodiments, operates independently of other messaging services such as SMS, as USSD, in some embodiments, relies on signaling channels rather than bearer channels. This allows for instantaneous (e.g., within less than 30 seconds, like less than 1 second, or less than 100 ms) communication without the delay associated with store-and-forward mechanisms. USSD, in some embodiments, also does not require the installation of additional software on the mobile device, as it may leverage native GSM capabilities typically supported by even the most basic feature phones.

Some embodiments may enhance the USSD protocol by implementing security measures, such as encryption at the application layer or two-factor authentication processes during the session. The protocol, in some embodiments, may also support integration with backend systems for complex workflows, such as retrieving user-specific data, recording transactions, or triggering automated actions in connected systems.

In some embodiments, diagnostic test results may be provided to patients via alternative channels or in combination with multiple channels to enhance accessibility and convenience. These channels may include SMS, voice calls (e.g., with text-to-speech models generating voice outputs and speech-to-text models transcribing inputs), native mobile applications, web portals, or other communication mechanisms, which may operate independently or in a complementary manner to ensure effective delivery of the results.

In other embodiments with SMS-based delivery, the diagnostic results may be transmitted as a structured text message to the patient's registered mobile number. The system may first encode the results using predefined templates or formats, potentially including identification markers such as test IDs or anonymized patient IDs. To enhance privacy, sensitive data may be obfuscated or truncated, requiring the patient to access additional information via a secured link or follow-up interaction. The SMS may also contain instructions or follow-up recommendations, such as scheduling a consultation. In some cases, encrypted links may be included, requiring users to authenticate to view detailed results.

In other embodiments with voice call-based delivery, the system may use an interactive voice response (IVR) platform to guide patients through the retrieval of their diagnostic results. Upon receiving a call or dialing a designated service number, patients may interact with automated prompts to authenticate their identity, such as entering a PIN or providing a verification code. The IVR system may then audibly present the results using synthesized speech or pre-recorded audio. In some embodiments, live operators may be available for additional explanation or support, particularly for complex or critical results.

In other embodiments, native mobile applications may provide a more interactive and feature-rich environment for accessing diagnostic results. Patients may download an application associated with the diagnostic service provider, allowing them to log in using secure credentials. The application may retrieve and display results in a structured, user-friendly format, potentially augmented by visual elements such as graphs, charts, or images (e.g., radiology scans). Push notifications may alert patients when new results are available, while the application may also offer options for secure storage, sharing with healthcare providers, or scheduling follow-ups.

Web portals in some embodiments may serve as another channel for result delivery, accessible via desktop or mobile web browsers. Patients may log into a secure online platform using credentials issued during their initial registration. Results may be displayed alongside additional contextual information, such as reference ranges, explanations of test parameters, or actionable recommendations. In some embodiments, the web portal may integrate with electronic health record (EHR) systems to allow seamless sharing with authorized medical professionals.

In hybrid approaches, results may be provided through combinations of these channels to accommodate varying patient preferences and technological access. For instance, a patient may receive an initial SMS notification that their results are available, along with instructions to access detailed information through a web portal or mobile application. Similarly, voice calls may supplement digital channels for patients who prefer or require verbal communication, while printouts of results could be made available at healthcare facilities for those with limited digital access.

To facilitate secure and reliable delivery, these channels may incorporate various safeguards, including encryption protocols, multi-factor authentication, and audit trails for access logs. Systems implementing these approaches may also support integration with translation services to deliver results in multiple languages or formats tailored to specific accessibility needs, such as text-to-speech for visually impaired users.

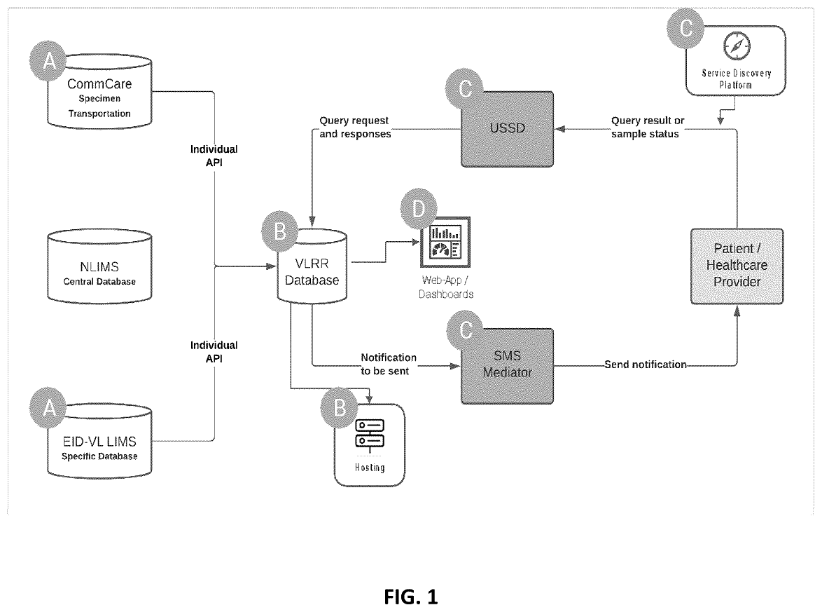

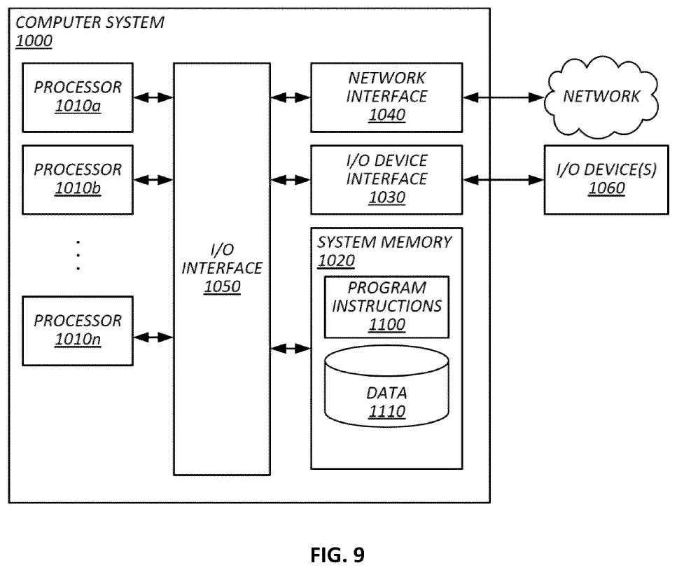

FIG. 1 depicts a block diagram of a computing environment consistent with some embodiments. The various components illustrated may, in some cases, be implemented using computer systems similar to those described in connection with FIG. 9. In some embodiments, the system may interact with a population of users'mobile computing devices (like more than 10, more than 100, or more than 10,000, distributed over a geographic area, like a metropolitan area, state, district, or country), which may include feature phones and smartphones. While the embodiment is described as a Viral Load Results Return (VLRR) application, it may also be configured to facilitate the return of various other types of diagnostic results.

Other types of diagnostic results that might be returned in some embodiments may include blood glucose levels for diabetes management; hemoglobin levels for anemia screening; cholesterol and lipid panel results for cardiovascular health monitoring; tuberculosis test results; malaria diagnostic test results, including rapid diagnostic test outcomes; human immunodeficiency virus test results, such as CD4 counts or antiretroviral therapy adherence markers; polymerase chain reaction results for pathogen detection, including viral load measurements for diseases other than HIV; complete blood count analysis, including white blood cell, red blood cell, and platelet counts; COVID-19 diagnostic test results, such as antigen or PCR test outcomes; cancer screening results, such as mammogram findings or Pap smear interpretations; genetic testing results, including predispositions to hereditary conditions; kidney function test results, such as creatinine and blood urea nitrogen levels; liver function test results, such as alanine aminotransferase and aspartate aminotransferase levels; infectious disease panel results, including sexually transmitted infection screenings; imaging-based diagnostic interpretations, such as ultrasound or X-ray summaries if digitized and translatable to text; allergy test results, including sensitivity profiles for environmental or food allergens; and prenatal test results, including markers for fetal health and maternal conditions. In some embodiments, the system may be configured to support returning results in a customizable format, depending on the specific medical condition, diagnostic test, or user requirements.

As illustrated by element A in FIG. 1, the integration of the Viral Load Results Return (VLRR) application with existing systems, including Specimen Transportation (CommCare) and the Early Infant Diagnosis/Viral Load Laboratory Information Management System (EID/VL LIMS), may be a component of the architectural design. Examples include a country's National Laboratory Information System (NLIMS) or a EID/VL LIMS or the Specimen Transportation system.

The Viral Load Results Return (VLRR) application may be designed to use both USSD and SMS technologies, integrated with existing data systems such as Result Transportation and the EID/VL LIMS. This, in some embodiments, facilitates the direct delivery of viral load results to patients and healthcare workers while also providing functionality to notify guardians of exposed infants when results are available at the health facility. Developed using C#, CSS, and Python, in some embodiments, the VLRR application offers a scalable solution capable of providing health results directly to patients at both national and global levels. It, in some embodiments, also facilitates two-way communication between healthcare workers and patients. By leveraging USSD and SMS, in some embodiments, the application ensures accessibility across all mobile phones, including feature phones and smartphones. This approach is particularly significant in Sub-Saharan Africa, where smartphone ownership is limited, especially in rural areas, allowing nearly anyone with access to a mobile phone to retrieve results. Users may include government health officials, patients, healthcare workers, and guardians of exposed infants.

Element D in FIG. 1 may include a VLRR back-end dashboard, which in some embodiments, is an administrative interface designed to track the total number of users, their activity status, and recent logins, with all data disaggregated by role and site. A user management component, in some embodiments, is included, allowing system administrators to add or edit site information, manage healthcare cadres and user roles, and disable inactive accounts as necessary. Built using C#, CSS, and Python, in some embodiments, the dashboard provides system administrators with the ability to modify the VLRR application or manage users as additional use cases are identified. The user-friendly interface, in some embodiments, allows for these modifications without requiring expertise in back-end coding. In some embodiments, the dashboard is accessible only to system administrators within a government health department, e.g., requiring password-protected access. In some embodiments, the dashboard ensures secure administrative control over the system.

As represented by element D in FIG. 1, an administrative dashboard has been established to support user management, tracking, and monitoring and evaluation functionalities. The dashboard is configured to track the total number of users, their activity status, and recent login information. These metrics are designed to be disaggregated by user role and site, enabling granular analysis and oversight.

Element D in FIG. 1 may include a VLRR Monitoring and Evaluations dashboard, which in some embodiments, is a real-time interface designed to track key indicators relevant to assessing the success of the VLRR application. Data, in some embodiments, is automatically pulled into the dashboard daily through integrated systems, ensuring that information is up-to-date and facilitating timely recommendations and adjustments to the application as needed. Developed using C#, CSS, and Python, in some embodiments, the dashboard provides a front-end visualization of statistics related to the return of viral load results to patients and healthcare workers. Users, in some embodiments, can view disaggregated data and explore more granular trends for indicators of interest. By consolidating information from multiple data sources into a single, cohesive interface, the dashboard, in some embodiments, is both easy to navigate and effective for monitoring purposes. Access may be password protected and limited, in some cases, to healthcare workers and government health officials. In some embodiments, the dashboard includes user management controls to add or remove users based on operational needs, ensuring accessibility while maintaining secure oversight.

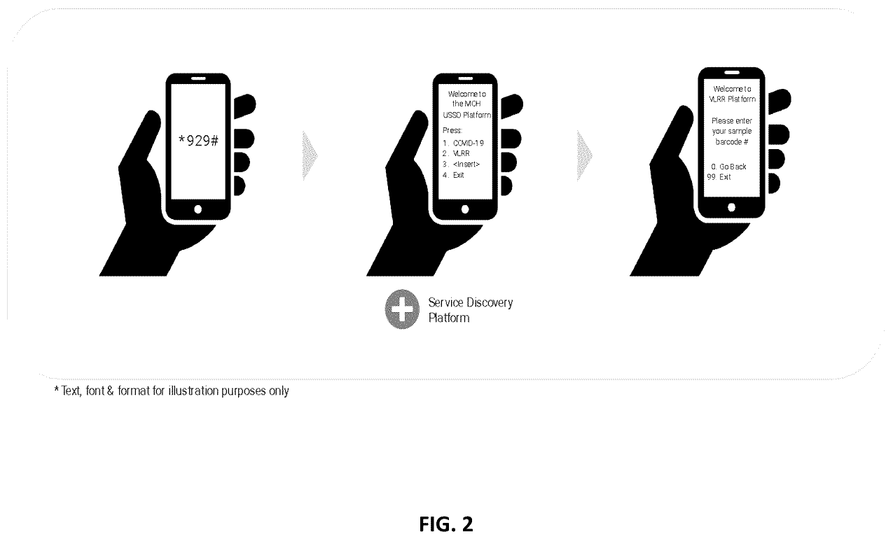

Some embodiments implement a Service Discovery Platform, like that described below with reference to FIG. 2, which may be designed to allow multiple products to share a single USSD short code (or each may have its own code). By dialing a single short code, such as 929, in some embodiments, users can access a variety of health-related data products, including services for COVID-19 and VLRR. Once a user selects an option, in some embodiments, they are seamlessly routed to the corresponding application. Built using C#, CSS, and Python, in some embodiments, the platform simplifies access for users, who no longer need to remember multiple short codes for individual services. For a Ministry of Health (MOH), in some embodiments, the platform reduces costs and time associated with applying for and maintaining separate short codes for each application. Additionally, since the short code is offered free of charge, in some embodiments, the system promotes sustainability by facilitating the addition of new applications without significant overhead.

Some embodiments include a Grafana VLRR Monitoring Application, which in some embodiments, is a multi-platform, open-source analytics and interactive visualization web application designed to provide charts, graphs, and alerts through the web when connected to supported data sources. Developed using Go and TypeScript, in some embodiments, it works in conjunction with an application called Prometheus, which collects metrics from various components of the VLRR system and feeds them to Grafana for visualization. This integration allows continuous monitoring of the VLRR system, in some embodiments, facilitating rapid responses to potential issues before the application goes offline or immediately upon detection of downtime.

Some embodiments have access to CommCare, which manages logistics information for viral load and early infant diagnosis specimens, and to EID/VL LIMS via an application program interface (API) connection, facilitating the transfer of data to the VLRR database.

As represented by element B in FIG. 1, the VLRR database and hosting infrastructure may be designed to comply with government requirements mandating that patient-level information be hosted on-premises. Some embodiments may have a dedicated server to host the application in a government mandated or approved location for the server, such as in-country.

As depicted by element C in FIG. 1, the USSD and Service Discovery Platform, along with the SMS Gateway, may be components of the VLRR application. A dedicated short code may be used to facilitate the USSD and SMS functionalities, such as one registered with a government authority or other authority managing a name space for such short codes (e.g., less than 10 characters, like less than 5, such as a 3 digit code).

Systems sharing a short codes may warrant inclusion of a service discovery platform. This platform, in some embodiments, routes users to various health services through a single short code. An example of such functionality is summarized in FIG. 2, and the platform may be configured to dynamically direct clients to specific applications or products associated with the short code. It is expected that the service discovery platform will provide, in some embodiments, long-term benefits by allowing new USSD-based services to be quickly configured and integrated into the platform.

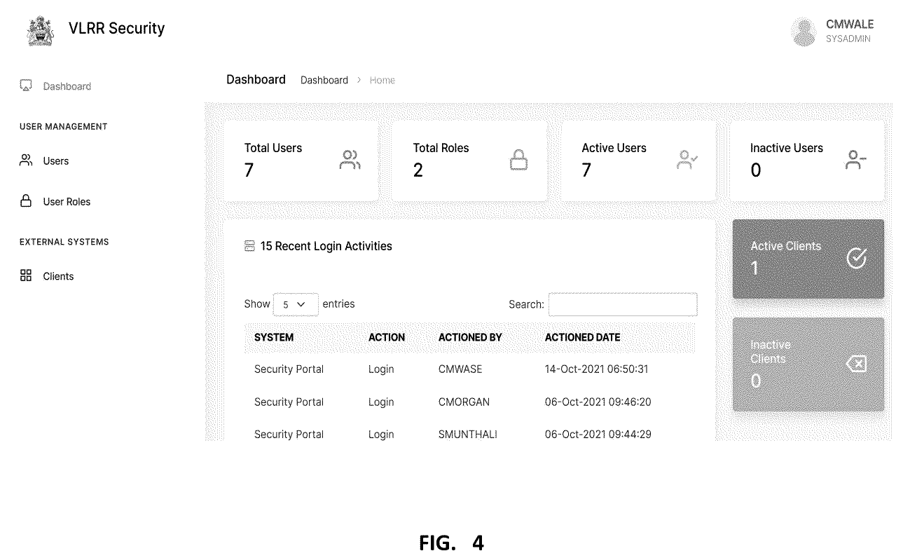

A user management component may also be implemented, allowing system administrators to perform tasks such as adding or editing site details, managing healthcare cadres and user roles, and disabling inactive accounts when necessary. An example visual representation of the home dashboard is provided in FIG. 4.

In some embodiments, several features may be incorporated to enhance the functionality and accessibility of the VLRR system. An opt-out feature may be introduced, allowing clients registered for VLRR to opt out of receiving SMS notifications that indicate their results are ready for viewing. This feature, in some embodiments, provides users with greater control over their communication preferences and privacy.

A PIN recovery option has also been added to the platform, in some embodiments. This feature allows clients, Healthcare Super Users (HCSUs), and Healthcare Providers to initiate the PIN recovery process by dialing a designated shortcode, *77. This, in some embodiments, ensures secure and convenient access to the platform in cases where a PIN has been lost or forgotten.

To address requests from healthcare workers (HCWs), a SMS alert feature may be implemented. This feature, in some embodiments, sends a list of Antiretroviral Therapy (ART) numbers with ready results directly to HCWs. By providing real-time notifications, in some embodiments, this feature ensures that HCWs are promptly informed when client results are available. For example, upon receiving an SMS with ART numbers, HCWs can review the results and identify those with high viral loads. For such cases, they can deploy an expert client into the community to follow up with the individual and facilitate their timely return to the facility for further management.

The VLRR Admin Dashboard has been implemented, in some embodiments, to include advanced features for improved monitoring and troubleshooting. A page may afford daily synchronization comparisons between results and Laboratory Information Management System (LIMS) data for the same facilities, aiding in data accuracy and consistency. Additionally, a data export feature may be implemented to expedite the identification and resolution of synchronization issues.

Some embodiments may include a security portal, configured to allow administrators to disable or enable HCSUs. This feature, in some embodiments, provides enhanced control over user access and role management, ensuring secure operation of the platform.

FIG. 2 illustrates the operation Service Discovery Platform associated with element C of FIG. 1. The USSD system may be designed to support three user roles: client, healthcare provider, and healthcare superuser. To enhance accessibility, the application may be configured to retrieve results in either English or Chichewa or other languages, accommodating language preferences.

Some embodiments support workflows in the USSD application include the following: retrieving results, which is accessible to clients, healthcare providers, and healthcare superusers; changing the language between English and Chichewa, available to all user roles; updating patient details to correct entry errors, a feature accessible to healthcare providers and healthcare superusers; and assigning, enabling, or deactivating a healthcare worker's access to a specific facility, a function reserved for healthcare superusers.

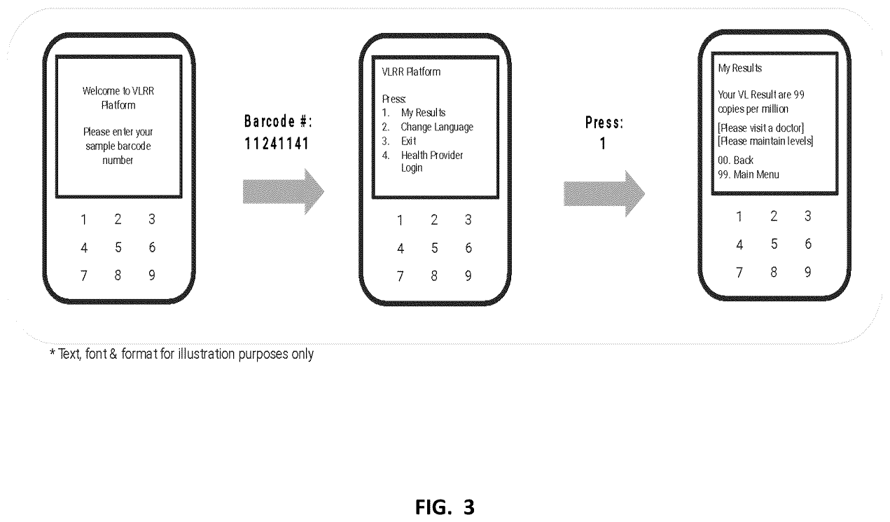

FIG. 3 depicts an example workflow for the USSD component. To establish the SMS mediator between the VLRR application and HCWs or clients, a gateway may be used to facilitate communication between the MOH systems and mobile network operators. Some embodiments may use a pre-configured service. Options for a pre-configured service included utilizing a local gateway or engaging a third-party vendor.

Some embodiments may implement a dedicated MOH SMS gateway, which could support potential scale-up and other future health-related products. The SMS mediator, in some embodiments, is configured to send generic notifications to clients or healthcare providers, informing them when a linked diagnostic result becomes available in the Viral Load/Early Infant Diagnosis (VL/EID) systems.

FIG. 4 illustrates the operation of element D in FIG. 1, specifically showcasing the Home Administration Dashboard. The Monitoring and Evaluation dashboard, in some embodiments, has been established to provide real-time tracking of key indicators for assessing the success of the VLRR application. Data, in some embodiments, is automatically aggregated into the dashboard on a daily basis through integration with connected systems. This frequent updating, in some embodiments, facilitates timely recommendations and adjustments to the application and facilitates rapid scaling if deemed necessary.

The homepage may include the following information: the total number of samples collected; the total number of results available; the number of users notified of result availability through SMS; the number of users who accessed results via the USSD application; and the average, minimum, and maximum number of days required to retrieve results.

All displayed metrics, in some embodiments, can be disaggregated by various parameters, including time period (e.g., last month, last three months), administrative level (e.g., national, district, site), area classification (e.g., rural, urban), and user role (e.g., client, healthcare worker). For each indicator, in some embodiments, users can download the corresponding raw dataset in formats such as XLSX or CSV, or save visual representations as images. Additionally, a “see more data trends” feature is provided for each indicator, in some embodiments, facilitating access to granular trend analysis on dedicated pages.

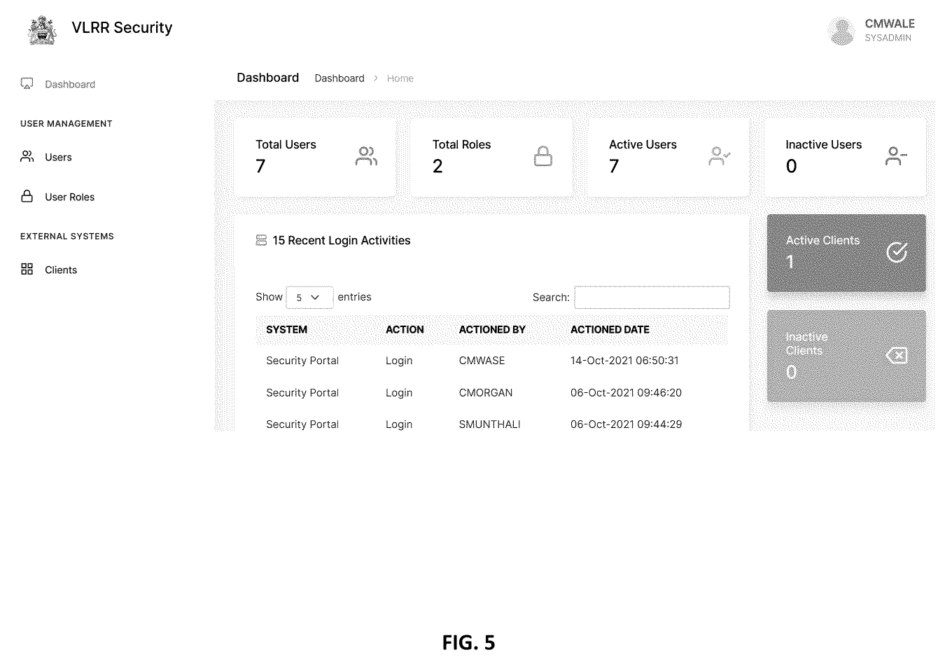

FIG. 5 illustrates an example design of a Home Monitoring and Evaluation Dashboard.

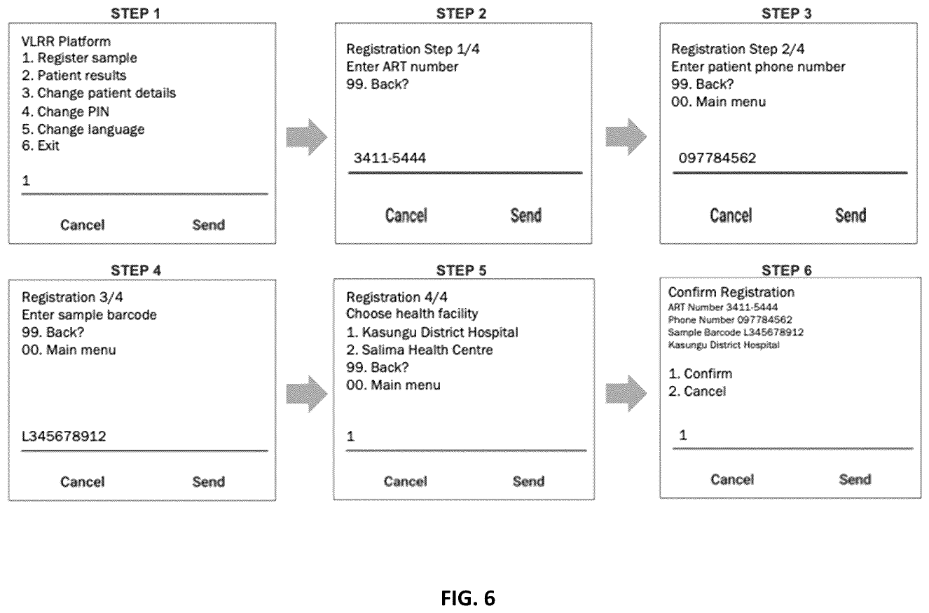

FIG. 6 illustrates an example USSD workflow for registering a client in the VLRR system. To establish the SMS mediator between the VLRR application and healthcare workers (HCWs) or clients, in some embodiments, as noted above, a gateway may be used to connect MOH systems with mobile network operators.

Some embodiments may implement a dedicated MOH-specific SMS gateway, which could support national scale-up and other future health-related products. The SMS mediator may be configured to send generic notifications to clients or HCWs whenever linked results became available in the Viral Load (VL) or Early Infant Diagnosis (EID) systems.

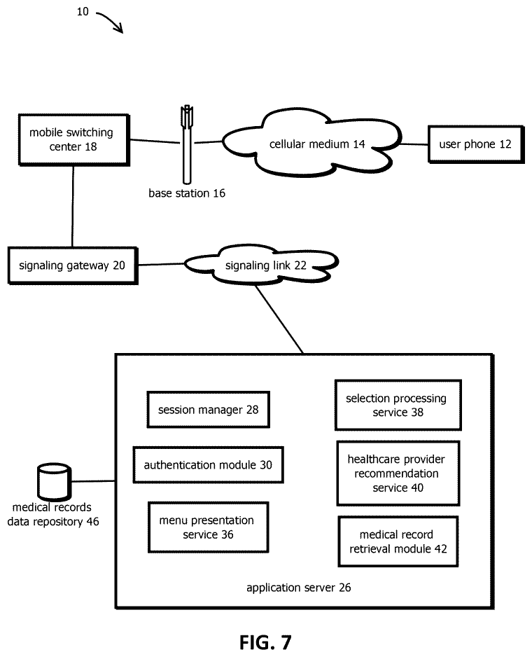

FIG. 7 is a block diagram of an example of a computing environment 10 in which some embodiments may be implemented. In some embodiments, the computing environment 10 may include a user phone 12 that may exchange signaling over a cellular wireless medium 14 with a base station 16, and the base station 16 may forward one or more signaling messages toward a mobile switching center 18. In some implementations, the mobile switching center 18 may route or relay at least a portion of that signaling through a signaling gateway 20 over one or more signaling links 22, and the signaling gateway 20 may communicate with an application server 26 that may host a session manager 28 and one or more service modules. In some cases, the computing environment 10 may support interactive exchanges in which a sequence of short user inputs from the user phone 12 may be mapped to corresponding server-generated text responses provided back to the user phone 12 through the same general signaling path.

In some embodiments, the user phone 12 may be a mobile device that may be capable of originating a service request using a dialer interface, and the user phone 12 may send the request without first establishing an Internet Protocol data session. In some implementations, the user phone 12 may be a feature phone that may omit an application runtime that executes third-party native applications, and the user phone 12 may still render server-provided text and accept user selections through keypad entry. In some cases, the user phone 12 may package a user-entered service code or session code into a control-plane message, and the user phone 12 may receive one or more corresponding control-plane responses that may carry menu text or other prompts. In some implementations, the user phone 12 may provide device identifiers, subscriber identifiers, or network-provided identifiers within the signaling exchange, and the user phone 12 may do so without exposing those identifiers to a third-party application layer on the device.

In some embodiments, the cellular wireless medium 14 may carry radio-layer control signaling between the user phone 12 and the base station 16, and the base station 16 may terminate at least a portion of a radio protocol stack. In some implementations, the cellular wireless medium 14 may include signaling associated with one or more cellular standards, and the signaling may include access requests, mobility management messages, and supplementary service invocation messages. In some cases, the base station 16 may forward received signaling toward the mobile switching center 18 through one or more wired or wireless backhaul links, and the forwarded signaling may be translated into a core-network signaling format. In some implementations, the base station 16 may attach metadata such as a serving cell identifier or location area information to the signaling, and the metadata may be used by server-side logic to tailor menu content or recommendations.

In some embodiments, the mobile switching center 18 may act as a switching and signaling node that may receive a service request originated by the user phone 12 and forwarded by the base station 16. In some implementations, the mobile switching center 18 may determine a destination for the service request based on a service code, a subscriber profile, a carrier routing rule, or a network configuration. In some cases, the mobile switching center 18 may create or reference a session context associated with the service request, and the session context may include a session identifier and one or more timers. In some implementations, the mobile switching center 18 may route transaction-style signaling toward the signaling gateway 20, and the mobile switching center 18 may do so via the signaling link 22 using a link-layer and transport-layer arrangement configured for signaling traffic.

In some embodiments, the signaling link 22 may include a signaling transport that may carry control-plane transactions between the mobile switching center 18 and the signaling gateway 20. In some implementations, the signaling link 22 may include a Signaling System No. 7 (SS7) linkset, and the signaling may include transaction messages that may carry user-visible text payloads and user responses. In some cases, the signaling link 22 may include an Internet Protocol transport that may encapsulate SS7 signaling using a signaling transport adaptation arrangement, and the encapsulation may be carried over a transport such as Stream Control Transmission Protocol (SCTP). In some implementations, the signaling link 22 may include redundancy, and the signaling link 22 may be implemented with multiple physical or logical paths and failover routing rules.

In some embodiments, the signaling gateway 20 may provide protocol interworking between a carrier signaling network and an Internet Protocol network that may be used to reach the application server 26. In some implementations, the signaling gateway 20 may receive SS7 signaling units from the mobile switching center 18 and may translate or map those signaling units into an application-facing request format. In some cases, the signaling gateway 20 may preserve a transaction identifier, a session identifier, or a dialogue identifier from the received signaling, and the signaling gateway 20 may forward that identifier to the application server 26 so that responses may be associated with the correct session. In some implementations, the signaling gateway 20 may perform address translation, message screening, rate limiting, or payload normalization prior to forwarding a request to the application server 26. In some cases, the signaling gateway 20 may also perform the reverse mapping for responses, where the signaling gateway 20 may receive a response payload from the application server 26 and may package the response into a signaling message suitable for delivery back through the mobile switching center 18 to the user phone 12.

In some embodiments, the application server 26 may be implemented as one or more computing systems that may execute server-side logic for interactive sessions, and the application server 26 may be reachable from the signaling gateway 20 over one or more packet networks. In some implementations, the application server 26 may expose an interface that may accept inbound session events, and the interface may represent events such as session initiation, user input, session continuation, and session termination. In some cases, the application server 26 may maintain per-session state in volatile memory, and the per-session state may include a current menu identifier, one or more authentication flags, a partially completed workflow, or a selected record identifier. In some implementations, the application server 26 may omit persistent storage of message bodies associated with a session, and the application server 26 may instead store only transient state and derived values that may expire based on time or based on a session termination signal.

In some embodiments, the session manager 28 may coordinate lifecycle management for interactive message exchanges, and the session manager 28 may allocate, update, and release session state entries. In some implementations, the session manager 28 may create a session record in memory upon receiving a session start indication from the signaling gateway 20, and the session record may include a session identifier and an expiration time. In some cases, the session manager 28 may treat inbound user inputs as events that may be processed synchronously while a carrier-side session remains active, and the session manager 28 may treat an inactivity period as a condition that may trigger session cleanup. In some implementations, the session manager 28 may maintain a mapping between a subscriber identifier and an active session identifier, and the mapping may be stored in a volatile cache with a time-to-live value. In some cases, the session manager 28 may refrain from storing complete transcripts of the user-visible prompts and user replies, and the session manager 28 may instead store a compact representation such as a menu state token, a workflow step index, or a pointer to a currently selected option.

In some embodiments, ephemeral message handling may be implemented by treating user-visible prompts as session-scoped payloads that may be generated and delivered during an active signaling dialogue and that may cease to be available when the dialogue ends. In some implementations, the computing environment 10 may deliver user-visible text through a live signaling channel that may be terminated by the network, by the user phone 12, or by the application server 26, and the text may not be persisted by the application server 26 after termination. In some cases, the computing environment 10 may omit store-and-forward message queuing associated with Short Message Service (SMS) delivery, and the computing environment 10 may avoid writing message bodies into a message center database as part of the interactive exchange. In some implementations, the computing environment 10 may still support store-and-forward delivery as an alternate path, and the same application server 26 may be configured to package a prompt as an SMS payload when a session-based signaling dialogue is unavailable or when carrier policy indicates SMS delivery. In some cases, the computing environment 10 may allow interactive retrieval and display of results on the user phone 12 without requiring an operating system on the user phone 12 that supports installation of third-party native applications, and the computing environment 10 may allow that interaction without requiring Internet access on the user phone 12 when signaling coverage is present.

In some embodiments, an authentication module 30 may process a session event stream and may decide whether a session is authenticated prior to releasing protected content. In some implementations, the authentication module 30 may request an authentication factor by causing the menu presentation service 36 to send a prompt that requests a personal identification number, a date of birth fragment, a passphrase, or a one-time code. In some cases, the authentication module 30 may verify an entered value by comparing a derived value to a stored verifier, where the derived value may be computed by applying a one-way transformation to the entered value and optionally a salt value. In some implementations, the authentication module 30 may use network-provided identity signals, and the authentication module 30 may compare a calling line identity or subscriber identifier provided in signaling to an expected identifier associated with a medical record. In some cases, the authentication module 30 may request step-up authentication, and the authentication module 30 may trigger delivery of a one-time code through an out-of-band channel such as voice, Short Message Service (SMS), or email when such channels are available. In some implementations, the authentication module 30 may update session state maintained by the session manager 28 to mark a session as authenticated for a limited duration, and the duration may be stored as a time-to-live value that may expire without further action.

In some embodiments, a menu presentation service 36 may generate user-visible prompts that may be transported through the signaling gateway 20 and rendered by the user phone 12, and the prompts may represent a structured menu. In some implementations, the menu presentation service 36 may format a menu as plain text that includes numbered options, and the numbering may be selected to match keypad inputs supported by the user phone 12. In some cases, the menu presentation service 36 may tailor menu content based on a language preference, a carrier-provided locale signal, or a prior selection stored in session state. In some implementations, the menu presentation service 36 may embed a compact state indicator within a server-side session record rather than embedding state within a long payload, and the menu presentation service 36 may rely on the session manager 28 to correlate subsequent user replies to the correct menu state. In some cases, the menu presentation service 36 may split a long prompt into multiple sequential payloads based on a payload size limit imposed by a signaling channel, and the menu presentation service 36 may mark each segment with an ordering indicator stored in session state.

In some embodiments, a selection processing service 38 may receive user input captured by the signaling path and may interpret that input as a selection, a free-form value, or a navigation command. In some implementations, the selection processing service 38 may parse an inbound payload to extract digits, separators, and control characters, and the selection processing service 38 may normalize the extracted input into a canonical form. In some cases, the selection processing service 38 may validate an input against a set of acceptable responses for the current menu state, and the acceptable responses may be stored as a set of allowed tokens associated with the menu identifier in session state. In some implementations, the selection processing service 38 may update session state in the session manager 28 to reflect a new workflow step, and the selection processing service 38 may trigger the menu presentation service 36 to send a next prompt based on the updated state. In some cases, the selection processing service 38 may detect an invalid input or a timeout, and the selection processing service 38 may cause a corrective prompt to be returned and may keep the session active for a bounded period.

In some embodiments, a medical record retrieval module 42 may receive a request determined from a user selection and may retrieve a corresponding medical record or a portion of a medical record from a medical records data repository 46. In some implementations, the medical record retrieval module 42 may translate a session-scoped record identifier into a repository query, and the query may specify a patient identifier, an encounter identifier, a test order identifier, or a result identifier. In some cases, the medical record retrieval module 42 may apply access controls prior to executing a query, and the access controls may be derived from authentication state produced by the authentication module 30 and from policy rules associated with the record. In some implementations, the medical record retrieval module 42 may fetch only a subset of fields that may be displayable within a constrained text payload, and the medical record retrieval module 42 may compute a short summary string from retrieved fields by selecting, truncating, and ordering values. In some cases, the medical record retrieval module 42 may redact or mask selected fields by replacing characters with placeholder symbols, and the selection of fields for redaction may depend on session authentication level, user role, or a jurisdiction rule stored as configuration. In some implementations, the medical record retrieval module 42 may cache derived summaries in volatile memory for a short period, and the cache entry may be keyed by a session identifier so that a repeat request within the same session may be served without re-querying the medical records data repository 46.

In some embodiments, the medical records data repository 46 may store structured and unstructured medical data, and the repository 46 may include one or more databases or storage systems. In some implementations, the repository 46 may store records in a relational database, and the repository 46 may store additional content in an object store that may be referenced by pointers in relational tables. In some cases, the repository 46 may store encrypted values at rest, and the medical record retrieval module 42 may decrypt selected values in memory prior to producing a user-visible payload. In some implementations, the repository 46 may expose an interface that supports queries by record identifier, patient identifier, or date range, and the interface may accept queries from the application server 26 through a private network connection. In some cases, the repository 46 may maintain audit metadata for accesses, and the audit metadata may be generated by the application server 26 when a retrieval is performed.

In some embodiments, a healthcare provider recommendation service 40 may generate a recommendation payload that may be delivered to the user phone 12 through the same session-based messaging path. In some implementations, the healthcare provider recommendation service 40 may accept a context that includes a user-entered location, a network-derived approximate location associated with the base station 16, a specialty selection, or an insurance plan selection. In some cases, the healthcare provider recommendation service 40 may query one or more provider directories, and the directories may be hosted within the computing environment 10 or accessed through a network interface exposed by the application server 26. In some implementations, the healthcare provider recommendation service 40 may compute a ranked list by assigning scores to candidate providers based on distance, availability windows, accepted insurance, language, or prior user preferences stored for the session, and the service 40 may format a top subset into short text lines that may fit within a constrained payload. In some cases, the healthcare provider recommendation service 40 may generate follow-up prompts that allow the user phone 12 to request additional details, and the selection processing service 38 may route such follow-up selections back to the healthcare provider recommendation service 40 to retrieve the next portion of the ranked list.

In some embodiments, the application server 26 may coordinate among the authentication module 30, the menu presentation service 36, the selection processing service 38, the healthcare provider recommendation service 40, and the medical record retrieval module 42 through internal message passing or function calls. In some implementations, the application server 26 may implement an event loop that processes inbound session events received from the signaling gateway 20, and the event loop may dispatch events to a handler based on a session state value maintained by the session manager 28. In some cases, the application server 26 may generate outbound payloads that are constrained to a target character limit, and the application server 26 may adapt content length by abbreviating labels, truncating values, or splitting content across sequential prompts. In some implementations, the application server 26 may provide observability signals such as metrics or logs that describe session durations and error types, and the observability signals may omit user-visible payload bodies to maintain ephemerality of the interactive messages.

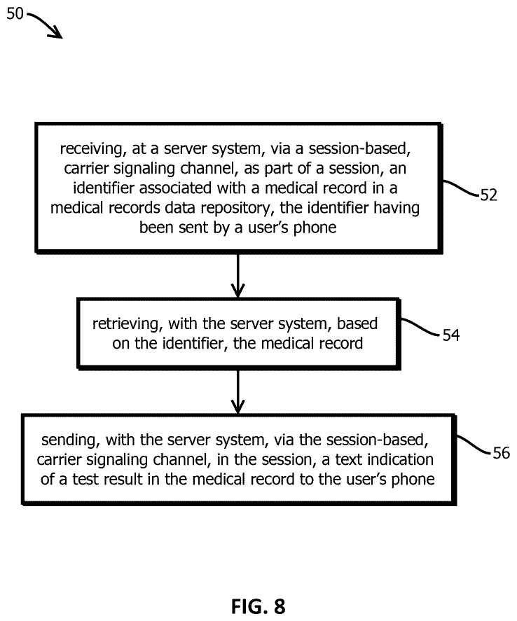

FIG. 8 illustrates an example of a process 50 that may be executed by the application server 26 described above or by other systems. In some embodiments, the process includes receiving at a server system via a session-based carrier signaling channel as part of a session, an identifier associated with a medical record and a medical records data repository where the identifier has been sent by a user's phone as indicated by block 52. Some embodiments may retrieve with the server system based on the identifier, the medical record from the medical records data repository as indicated by block 54. Some embodiments may send with the server system via the session-based carrier signaling channel in the session a text indication of a test result in the medical record to the user's phone as indicated by block 56. The test result may be expressly stated in the medical record or it may be inferred from the medical record.

FIG. 9 is a diagram that illustrates an exemplary computing system 1000 in accordance with embodiments of the present technique. A single computing device is shown, but some embodiments of a computer system may include multiple computing devices that communicate over a network, for instance in the course of collectively executing various parts of a distributed application. Various portions of systems and methods described herein, may include or be executed on one or more computer systems similar to computing system 1000. Further, processes and modules described herein may be executed by one or more processing systems similar to that of computing system 1000.

Computing system 1000 may include one or more processors (e.g., processors 1010a-1010n) coupled to system memory 1020, an input/output I/O device interface 1030, and a network interface 1040 via an input/output (I/O) interface 1050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 1020). Computing system 1000 may be a uni-processor system including one processor (e.g., processor 1010a), or a multi-processor system including any number of suitable processors (e.g., 1010a-1010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 1000 may include a plurality of computing devices (e.g., distributed computer systems) to implement various processing functions.

I/O device interface 1030 may provide an interface for connection of one or more I/O devices 1060 to computer system 1000. I/O devices may include devices that receive input (e.g., from a user) or output information (e.g., to a user). I/O devices 1060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 1060 may be connected to computer system 1000 through a wired or wireless connection. I/O devices 1060 may be connected to computer system 1000 from a remote location. I/O devices 1060 located on remote computer system, for example, may be connected to computer system 1000 via a network and network interface 1040.

Network interface 1040 may include a network adapter that provides for connection of computer system 1000 to a network. Network interface may 1040 may facilitate data exchange between computer system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may be configured to store program instructions 1100 or data 1110. Program instructions 1100 may be executable by a processor (e.g., one or more of processors 1010a-1010n) to implement one or more embodiments of the present techniques. Instructions 1100 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 1020 may include a non-transitory computer readable storage medium that may have program instructions stored thereon that are executable by a computer processor (e.g., one or more of processors 1010a-1010n) to cause the subject matter and the functional operations described herein. A memory (e.g., system memory 1020) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may be configured to coordinate I/O traffic between processors 1010a-1010n, system memory 1020, network interface 1040, I/O devices 1060, and/or other peripheral devices. I/O interface 1050 may perform protocol, timing, or other data transformations to convert data signals from one component (e.g., system memory 1020) into a format suitable for use by another component (e.g., processors 1010a-1010n). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 1000 or multiple computer systems 1000 configured to host different portions or instances of embodiments. Multiple computer systems 1000 may provide for parallel or sequential processing/execution of one or more portions of the techniques described herein.

Those skilled in the art will appreciate that computer system 1000 is merely illustrative and is not intended to limit the scope of the techniques described herein. Computer system 1000 may include any combination of devices or software that may perform or otherwise provide for the performance of the techniques described herein. For example, computer system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 1000 may be transmitted to computer system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, e.g., text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

The present techniques will be better understood with reference to the following enumerated embodiments:
1. A method, comprising: receiving, at a server system, via a session-based, carrier signaling channel, as part of a session, an identifier associated with a medical record in a medical records data repository, the identifier having been sent by a user's phone; retrieving, with the server system, based on the identifier, the medical record; and sending, with the server system, via the session-based, carrier signaling channel, in the session, a text indication of a test result in the medical record to the user's phone.
2. The method of embodiment 1, wherein receiving the identifier comprises receiving, from the user's phone, a response to a text menu prompt transmitted in the session via the session-based, carrier signaling channel.
3. The method of embodiment 1, further comprising, before retrieving the medical record, authenticating the user by receiving, in the session, an authentication value comprising at least one of a personal identification number (PIN) or a one-time passcode (OTP), and permitting the retrieving based on successful authentication.
4. The method of embodiment 1, wherein the session is initiated by a service-code request entered at the user's phone and routed by a cellular carrier signaling network to the server system.
5. The method of embodiment 1, wherein the identifier associated with the medical record comprises at least one of (i) a patient identifier, (ii) an accession number, (iii) a specimen identifier, or (iv) a treatment program identifier.
6. The method of embodiment 1, further comprising receiving, at the server system via the session-based, carrier signaling channel, a device-associated identifier corresponding to the user's phone, and validating that the device-associated identifier is authorized to access the medical record identified by the identifier.
7. The method of embodiment 1, wherein sending the text indication of the test result comprises sending a limited-content indication that excludes at least one of a patient name, a full date of birth, or a diagnosis description.
8. The method of embodiment 1, further comprising, prior to sending the text indication of the test result, determining a role associated with the user and selecting between (i) sending the text indication of the test result to the user's phone and (ii) sending a text notification that the test result is available without including the test result.
9. The method of embodiment 1, further comprising terminating the session based on a timeout, and, in response to terminating the session, refraining from sending additional content via the session-based, carrier signaling channel until a new session is established.
10. The method of embodiment 1, further comprising writing, by the server system, an audit record indicating that the test result was sent in the session, the audit record including at least one of the identifier, a timestamp, or a device-associated identifier.
11. The method of embodiment 1, wherein retrieving the medical record comprises obtaining the medical record from the medical records data repository through an interface to a laboratory information management system or an electronic medical record system.
12. The method of embodiment 1, further comprising sending contact information for a healthcare provider to the user's phone based on the test result.
13. The method of embodiment 1, further comprising authenticating a patient associated with the medical record by receiving, via the session-based, carrier signaling channel and in the session, at least one authentication factor.
14. The method of embodiment 1, wherein receiving the identifier comprises receiving the identifier in a Session Initiation Protocol (SIP) message encapsulated in Internet Protocol (IP) packets and routed through a private IP network of a cellular carrier without routing through the public Internet.
15. The method of embodiment 1, wherein receiving the identifier comprises receiving the identifier via either (i) a Signaling System No. 7 (SS7) signaling link or (ii) a SIGTRAN signaling transport, the SS7 signaling link or the SIGTRAN signaling transport being associated with a mobile switching center of a cellular carrier.
16. The method of embodiment 1, further comprising exchanging a plurality of messages in the session between the server system and the user's phone via the session-based, carrier signaling channel to present at least one text prompt and receive at least one user selection.
17. The method of embodiment 1, wherein the session-based, carrier signaling channel is distinct from (i) an Internet data session used to exchange HTTP messages and (ii) a store-and-forward message service in which received messages are stored on the user's phone.
18. The method of embodiment 1, wherein the user's phone is a feature phone.
19. The method of embodiment 1, wherein the user's phone lacks an operating system configured to execute third-party native applications.
20. A computer-readable medium storing instructions that, when executed, effectuate operations comprising: the operations of any one of embodiments 1-19.
21. A system comprising one or more processors and memory storing instructions that when executed effectuate the operations comprising: the operations of any one of embodiments 1-19.

What is claimed is:

1. A method, comprising:
receiving, at a server system, via a session-based, carrier signaling channel, as part of a session, an identifier associated with a medical record in a medical records data repository, the identifier having been sent by a user's phone;
retrieving, with the server system, based on the identifier, the medical record; and
sending, with the server system, via the session-based, carrier signaling channel, in the session, a text indication of a test result in the medical record to the user's phone.

2. The method of claim 1, wherein receiving the identifier comprises receiving, from the user's phone, a response to a text menu prompt transmitted in the session via the session-based, carrier signaling channel.

3. The method of claim 1, further comprising, before retrieving the medical record, authenticating the user by receiving, in the session, an authentication value comprising at least one of a personal identification number (PIN) or a one-time passcode (OTP), and permitting the retrieving based on successful authentication.

4. The method of claim 1, wherein the session is initiated by a service-code request entered at the user's phone and routed by a cellular carrier signaling network to the server system.

5. The method of claim 1, wherein the identifier associated with the medical record comprises at least one of (i) a patient identifier, (ii) an accession number, (iii) a specimen identifier, or (iv) a treatment program identifier.

6. The method of claim 1, further comprising receiving, at the server system via the session-based, carrier signaling channel, a device-associated identifier corresponding to the user's phone, and validating that the device-associated identifier is authorized to access the medical record identified by the identifier.

7. The method of claim 1, wherein sending the text indication of the test result comprises sending a limited-content indication that excludes at least one of a patient name, a full date of birth, or a diagnosis description.

8. The method of claim 1, further comprising, prior to sending the text indication of the test result, determining a role associated with the user and selecting between (i) sending the text indication of the test result to the user's phone and (ii) sending a text notification that the test result is available without including the test result.

9. The method of claim 1, further comprising terminating the session based on a timeout, and, in response to terminating the session, refraining from sending additional content via the session-based, carrier signaling channel until a new session is established.

10. The method of claim 1, further comprising writing, by the server system, an audit record indicating that the test result was sent in the session, the audit record including at least one of the identifier, a timestamp, or a device-associated identifier.

11. The method of claim 1, wherein retrieving the medical record comprises obtaining the medical record from the medical records data repository through an interface to a laboratory information management system or an electronic medical record system.

12. The method of claim 1, further comprising sending contact information for a healthcare provider to the user's phone based on the test result.

13. The method of claim 1, further comprising authenticating a patient associated with the medical record by receiving, via the session-based, carrier signaling channel and in the session, at least one authentication factor.

14. The method of claim 1, wherein receiving the identifier comprises receiving the identifier in a Session Initiation Protocol (SIP) message encapsulated in Internet Protocol (IP) packets and routed through a private IP network of a cellular carrier without routing through the public Internet.

15. The method of claim 1, wherein receiving the identifier comprises receiving the identifier via either (i) a Signaling System No. 7 (SS7) signaling link or (ii) a SIGTRAN signaling transport, the SS7 signaling link or the SIGTRAN signaling transport being associated with a mobile switching center of a cellular carrier.

16. The method of claim 1, further comprising exchanging a plurality of messages in the session between the server system and the user's phone via the session-based, carrier signaling channel to present at least one text prompt and receive at least one user selection.

17. The method of claim 1, wherein the session-based, carrier signaling channel is distinct from (i) an Internet data session used to exchange HTTP messages and (ii) a store-and-forward message service in which received messages are stored on the user's phone.

18. The method of claim 1, wherein the user's phone is a feature phone.

19. The method of claim 1, wherein the user's phone lacks an operating system configured to execute third-party native applications.

20. A tangible, non-transitory, machine-readable medium storing instructions that, when executed by a computer system, cause the computer system to effectuate operations comprising:
receiving, at a server system, via a session-based, carrier signaling channel, as part of a session, an identifier associated with a medical record in a medical records data repository, the identifier having been sent by a user's phone;
retrieving, with the server system, based on the identifier, the medical record; and
sending, with the server system, via the session-based, carrier signaling channel, in the session, a text indication of a test result in the medical record to the user's phone.
